(19) United States
(12) Patent Application Publication
(10) Pub. No.: US 2016/0046898 A1
(43) Pub. Date: Feb. 18, 2016

(54) CELL GROWTH MACROCARRIERS FOR BIOREACTORS

(71) Applicant: PBS Biotech, Inc., Camarillo, CA (US)

(72) Inventors: C. Brian Lee, Thousand Oaks, CA (US); Yasunori Hashimura, Camarillo, CA (US)

(21) Appl. No.: 14/819,651

(22) Filed: Aug. 6, 2015

Related U.S. Application Data
(60) Provisional application No. 62/036,213, filed on Aug. 12, 2014.

(51) Int. Cl. C12M 1/12; C12M 1/00

(52) U.S. Cl. CPC C12M 25/16; C12M 23/20

(57) ABSTRACT

Macrocarriers for growing cells in bioreactors having generally ball-shaped outer profiles, with approximately the same dimension from side-to-side in all planes through the center of the macrocarrier. A series of generally horizontal plates and vertical ribs defined by the macrocarriers form flat surfaces for desirable cell growth.

### RELATED APPLICATION INFORMATION

This patent claims priority from the following provisional patent application: Provisional Patent Application No. 62/036,213, entitled Cell Growth Macrocarriers for Bioreactors, filed Aug. 12, 2014.

### TECHNICAL FIELD

The invention pertains to macrocarriers for bioreactors.

### BACKGROUND OF THE INVENTION

Efforts of biopharmaceutical companies to discover new biological drugs have increased exponentially in the past two decades. An increasing number of biological drug candidates are in development. For instance, pluripotent and multipotent stem cells have the potential to revolutionize various therapeutic applications, especially in the fields of regenerative medicine and pharmaceutical development.

Traditionally, anchorage-dependent animal cells have been cultured at lab scale on flat tissue culture plates and flasks whose surface has been pre-treated with special coating to facilitate cell attachment. For generating a large number of cells, this approach of growing cells only on a horizontal surface quickly becomes impractical, and a mechanism that utilizes the entire volume of a system such as a bioreactor becomes necessary.

Shortages of global biomanufacturing capacity are anticipated in the foreseeable future, particularly as production needs will increase as such new drugs are introduced to the market. Bioreactors have been used for cultivation of microbial organisms for production of various biological or chemical products. Most biological drugs are produced by cell culture or microbial fermentation processes which require sterile bioreactors and an aseptic culture environment.

A production bioreactor contains culture medium in a sterile environment that provides various nutrients required to support growth of the biological agents of interest. Stainless steel stirred tanks were originally used for large scale production of biological products in suspension culture. Such conventional bioreactors use mechanically driven impellors to mix the liquid medium during cultivation. The bioreactors can be reused for the next batch of biological agents after cleaning and sterilization of the vessel, which requires a significant amount of time and resources, especially to monitor and to validate each cleaning step prior to reuse for production of biopharmaceutical products. Due to the high cost of construction, maintenance and operation of the conventional bioreactors, single use bioreactor systems made of disposable plastic material have become an attractive alternative.

Microcarriers are cell attachment substrates used in culturing anchorage-dependent cells in a bioreactor. They are typically large enough to allow sufficient cell expansion upon attachment, but they are also small enough to provide a large surface area-per-volume ratio to support high cell density. Additionally, the size of microcarriers is small enough to allow good suspension in a traditional impeller-based bioreactor system at agitation rates that are low enough so as not to impart detrimental shearing effect on the cells. A typical size range for commercially available microcarrier beads is 75 to 250 micron in diameter. These microcarriers consist of either solid beads that permit cell attachment only on the surface or porous beads that permit cell attachment inside the beads, which offer the benefit of more surface area per bead volume for higher cell density processes as well as provide some level of mechanical protection from the shearing effects of the fluid motion if the cells are attached within the pores.

Although the most widely used carriers are microcarriers, macrocarriers for use in bioreactors have also been proposed. A general rule of thumb is that a microcarrier has a size up to the 500 μm range, while a macrocarrier is larger, though the terms are not always used consistently. For the purpose of the present application, a “macrocarrier” will be deemed to have a size of at least 1000 μm (1 mm) across its widest point.

Although there are a number of macrocarrier designs in the art, none as yet has the ability to grow high quality cells in large quantities.

### SUMMARY OF THE INVENTION

The present application discloses a number of macrocarriers for growing cells in bioreactors. Each macrocarrier has a generally ball-shaped outer profile, with approximately the same dimension from side-to-side in all planes through the center of the macrocarrier. At the same time, the macrocarriers define flat surfaces for desirable cell attachment and growth.

The embodiments of the present invention address the drawbacks of prior art microcarrier and macrocarrier designs and offer a solution for all of the challenges described above. By combining the porous bead concept that offers protection from mechanical shearing effects inside the bioreactor with flat surfaces to mimic the culture conditions of flat tissue culture flasks, this invention could help alleviate potential regulatory concerns in the cell therapy market while allowing high cell expansion.

The following objects of the invention set forth various desirable aspects that may be combined or embodied separately. In general, it is desirable that the macrocarriers:

Demonstrate suspendability under typical agitation environments (likely varies by vessel volume);

Maximize the surface area per liquid volume displaced;

Maximize the surface area per gram of material;

Maximize the cell accessibility to interior surfaces;

Minimize the resistance to liquid flow between stack plates or other interior volumes;

Have the ability to be coated with collagen or other compounds that facilitate cell attachment;

Have a geometry that minimizes the potential for nesting with adjacent macrocarriers.

An appreciation of the other aims and objectives of the present invention and an understanding of it may be achieved by referring to the accompanying drawings and the detailed description of a preferred embodiment.

### BRIEF DESCRIPTION OF THE DRAWINGS

FIGS. 1A-1D are perspective and orthogonal views of a first embodiment of a macrocarrier in a “Beach Ball” shape;

FIGS. 2A-2D are perspective and orthogonal views of a second embodiment of a macrocarrier in a “Jagged Equator” shape;

FIGS. 3A-3D are perspective and orthogonal views of a third embodiment of a macrocarrier in a “Honey Dipper” shape;

FIGS. 3E and 3F are vertical sectional views through the “Honey Dipper” macrocarrier of FIGS. 3A-3D taken along lines 3E-3E and 3F-3F of FIG. 3C; and

FIG. 4 is a perspective view of a small-volume bioreactor in which the macrocarriers of the present application can be utilized.

### DESCRIPTION OF THE PREFERRED EMBODIMENTS

The present application relates to cell macrocarriers for culturing biological cells, such as pluripotent or multipotent stem cells, wherein the carriers are suspended in a bioreactor. The macrocarrier may be modified by a surface treatment for better cell attachment, controlled growth and easy release. The surface treatment may include applying a coating material such as collagen, gas plasma treatment, corona discharge treatment or combinations thereof.

In the cell therapy field where cells are cultured and harvested as products, it is desirable that these cells grown in bioreactors are exposed to a similar growth environment as is provided by flat tissue culture plates used at lab scale in the early phase of process development. While the porous microcarriers that are currently available in the market help reduce the impact of shearing forces from the agitator that cells grown on flat tissue culture plates do not experience, the applicants have noticed that the cells oftentimes do not get embedded deep inside the pores due to the limited pore size and end up growing in multi-layer clumps on the surface of the microcarrier. These clumps, if large enough, create an environment for the cells whereby the cell population is exposed to significantly different levels of nutrients, growth factors, and oxygen content in the medium, increasing the likelihood that they result in a non-uniform population of both undifferentiated and partially differentiated cell types.

Furthermore, cells cultured on non-flat, curved surfaces such as on the surface of or inside the pores of the microcarriers often appear visually to have much different morphology than those cultured on flat tissue culture plates, and there may be some challenges that cell therapy developers face in demonstrating equivalent efficacy and identity of cells produced during volumetric scale up of process from flat stock to microcarriers in bioreactors. In short, cells cultured on flat surfaces are considered the gold-standard, but until now large-scale propagation of such cells has been time-consuming and relatively expensive.

Lastly, existing microcarriers that are available commercially are typically made in a way that does not allow for a tight particle size distribution during manufacture, resulting in significant variability in particle size. In the case of the one maker of microcarriers, for instance, the difference in diameter of particles in the 5^{th} percentile to 95th percentile is 101 micron even though the mean diameter is only 190 micron. This large variability in particle size is undesirable from a cell culture standpoint, as it may affect the dynamics of microcarrier suspension, the kinetics of cell attachment to microcarriers, and the rate at which cells reach confluence on microcarriers, all of which are important parameters to understand and control in adherent cell culture processes.

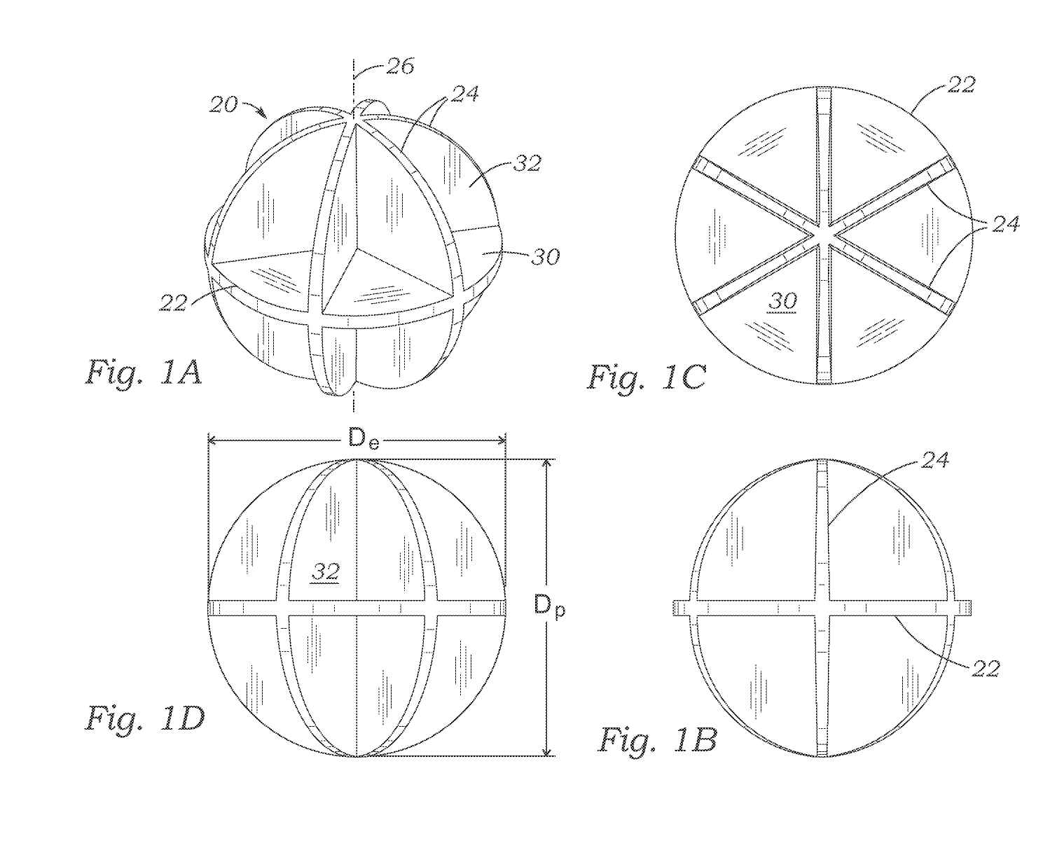

The macrocarriers disclosed herein are generally spherical or ball-shaped beads, but with multiple layers of flat surfaces inside each bead to maximize surface area, as exemplified by the embodiments described below (FIGS. 1-3). The flat surfaces are formed by a number of plates (generally horizontal) and ribs (generally vertical). To allow efficient transport of cells into the interstices between the plates and ribs and prevent this rate from becoming diffusion-limited, there is preferably sufficient distance between layers (100+ microns). Consequently, the particle size could also be large enough (2+ mm) to use injection molding as a preferred mode of manufacture, which would greatly reduce particle size tolerance that is problematic with existing microcarrier design.

With reference now to FIGS. 1A-1D, a first embodiment of a macrocarrier 20 somewhat resembles a “Beach Ball” in configuration. As mentioned above, the macrocarrier 20 has a spherical outer profile or external surface of revolution. In general, the macrocarriers described herein are generally spherical or ball-shaped beads formed by a series of plates and ribs with spaces or interstices therebetween, meaning that their surfaces of revolution have approximately the same dimension from side-to-side in all planes through the center of the macrocarrier. A purely spherical surface of revolution is synonymous with multi-axi-symmetric, while an oval-shaped surface of revolution is axisymmetric only about a polar axis along the long dimension.

FIG. 1D indicates an exemplary polar diameter Dₚ and equatorial diameter Dₑ, which are preferably equal, but could be slightly dissimilar so as to define an oval-shaped surface of revolution. For example, Dₚ>Dₑ to result in an oval-shaped surface of revolution which is axi-symmetric about the central vertical axis. In a preferred embodiment, both the polar diameter Dₚ and equatorial diameter Dₑ are within the range of 1-5 mm, preferably between 2-3 mm. It should be noted that the dimensional ranges noted for the macrocarrier 20 apply to the other macrocarriers described herein, and thus will not be repeated.

The macrocarrier 20 comprises a single horizontal plate 22 in the equatorial plane, and a plurality of vertical ribs 24 angularly spaced from each other and each intersecting a central vertical axis 26. In the illustrated embodiment, there are three vertical ribs 24 equally spaced around the vertical axis 26 at 60° angles from each other. With the equatorial plate 22, this creates twelve wedge-shaped spaces or interstices within the external profile of the macrocarrier 20 and between the plates 22 and ribs 24—six below and six above the equatorial plate 22. A larger number of vertical ribs 24 may be utilized, up to a practical limit that still allows efficient transport of cells into the interstices between the plates and ribs without becoming diffusion-limited. For example, desirably there is a distance between vertical ribs 24 of at least 100 microns. Preferably, there are at least three vertical ribs 24 as shown, up to about 30 ribs.

As was mentioned, the “Beach Ball” macrocarrier 20 provides a plurality of flat surfaces within its exterior contours that provide excellent terrain for growing a variety of species of cells in bioreactors. In particular, the horizontal plate 22 provides a number of generally triangular horizontal surfaces 30 on both top and bottom faces. The vertical ribs 24 likewise provide a plurality of generally triangular vertical surfaces 32 on each face thereof. In the illustrated embodiments, there are twelve horizontal surfaces 30 and twenty-four vertical surfaces 32.

The macrocarrier 20 may be formed from a variety of materials, preferably moldable polymers such as polystyrene (PS), polyethylene (PE), polycarbonate (PC), and polypropylene (PP). The flat surfaces 30, 32 may be treated so as to enhance cell growth. For example, the macrocarrier 20 may be immersed in a collagenous solution prior to use so as to coat the flat surfaces 30, 32 with collagen. Some cell growth processes, however, require the absence of any animal cell components, in which case the flat surfaces 30, 32 may be roughened somewhat using gas plasma treatment, corona discharge treatment or combinations thereof. It should be understood that each of the various macrocarriers described herein can be treated in the same manner, and thus this explanation will not be repeated.

The vertical ribs 24 of the “Beach Ball” macrocarrier 20 also exhibit a nonuniform thickness. As seen best in FIG. 1B, each of the vertical ribs 24 is thickest at the equatorial plane and gradually becomes thinner toward the poles.

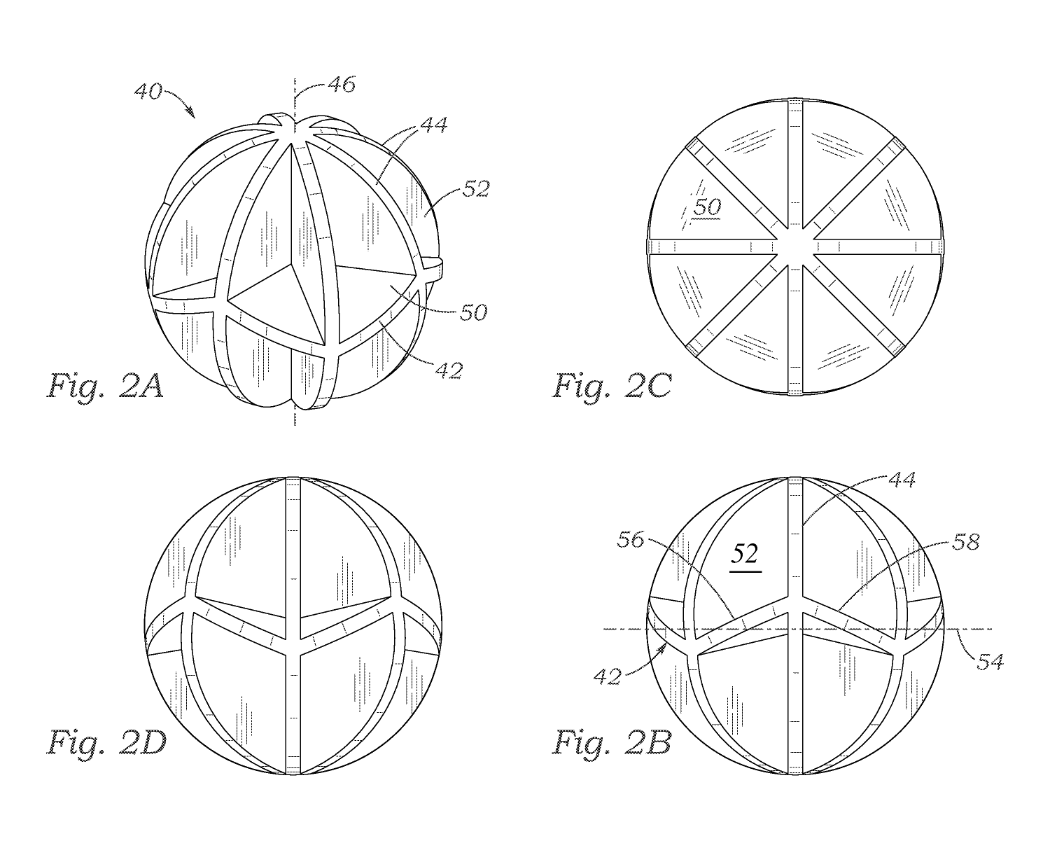

FIGS. 2A-2D illustrates a second embodiment of a macrocarrier 40 that has a “Jagged Equator” configuration. Like the “Beach Ball” concept, the macrocarrier 40 has a spherical outer profile or external surface of revolution.

The macrocarrier 40 comprises a single non-planar equatorial plate 42, and a plurality of planar vertical ribs 44 angularly spaced from each other and each intersecting a central vertical polar axis 46. In the illustrated embodiment, there are four planar vertical ribs 44 equally spaced around the vertical axis 46 at 45° angles from each other. With the equatorial plate 42, this creates sixteen wedge-shaped spaces or interstices within the external profile of the macrocarrier 40 and between the plate 42 and ribs 44—eight below and eight above the equatorial plate 42. Internal surfaces created by the plate 42 and ribs 44 are flat. More particularly, a series of flat, angled generally triangular surfaces 50 are formed on the top and bottom faces of the equatorial plate 42, while a number of generally triangular vertical surfaces 52 are formed on either side of the vertical ribs 44.

Again, a larger number of vertical ribs 44 may be utilized, up to a practical limit that still allows efficient transport of cells into the interstices between the plates and ribs without becoming diffusion-limited. For example, there are at least four vertical ribs 44 as shown, up to about 30 ribs.

In contrast with the first embodiment, the “Jagged Equator” macrocarrier 40 has a non-planar equatorial plate 42 that alternates angular orientation in the spaces between each vertical rib 44. For example, as seen in FIG. 2B, a horizontal equatorial plane 54 is shown bisecting the vertical rib 44 that is oriented directly perpendicular to the plane of the page. The equatorial plate 42 alternates between first segments 56 that are angled downward in a clockwise direction (looking down) around the equator and second segments 58 that are angled upward in a clockwise direction. This results in a “Jagged Equator” configuration for the equatorial plate 42. Although the segments 56, 58 are angled, they remain flat and thus produce desirable cells thereon.

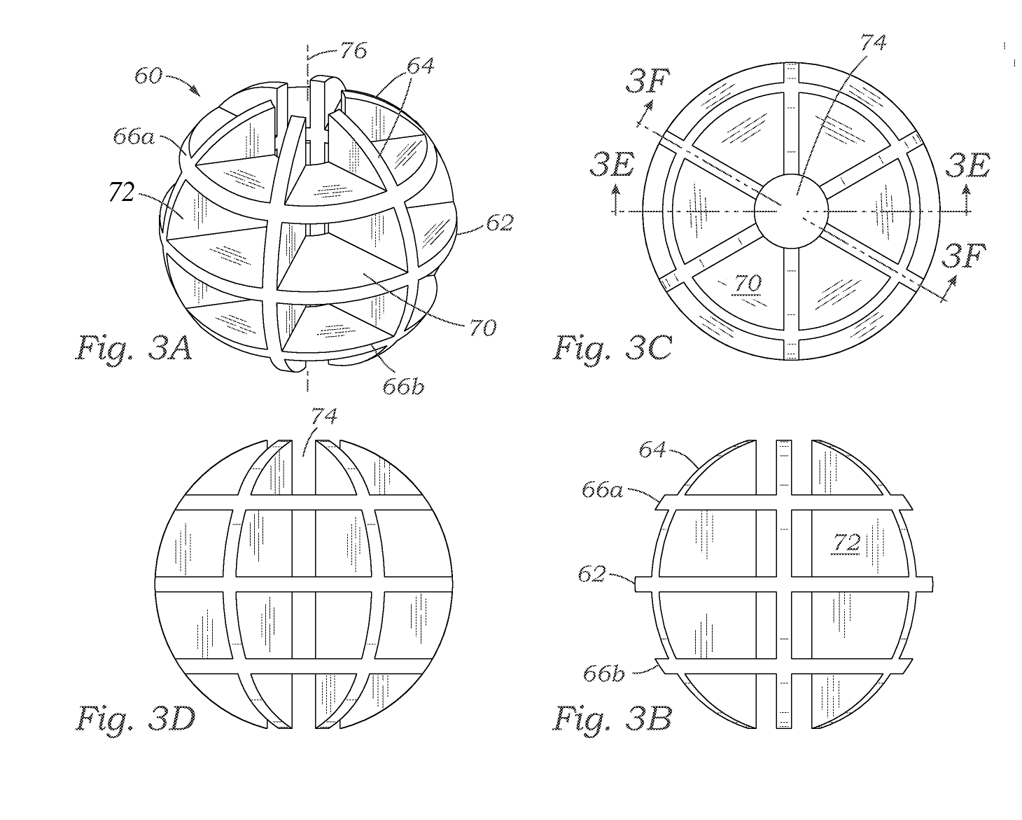

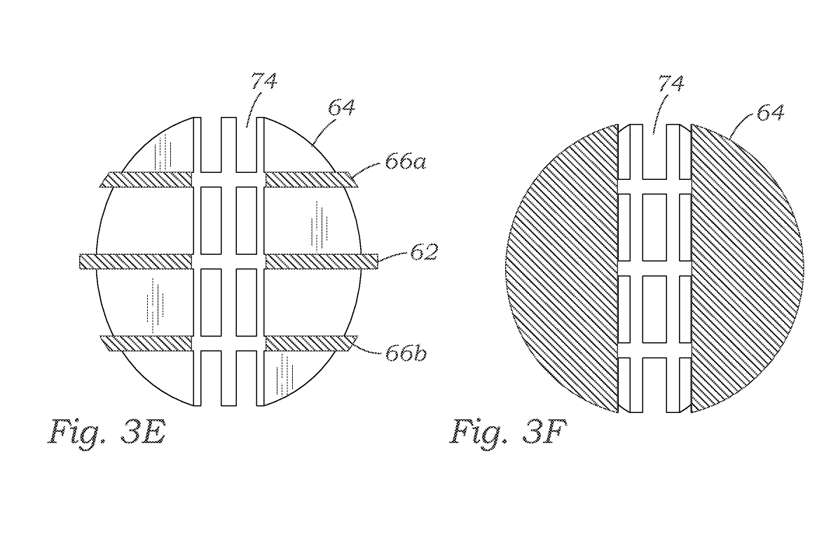

Now with reference to FIGS. 3A-3F, a third embodiment of a macrocarrier 60 is shown which is termed the “Honey Dipper,” but could also be viewed as a globe with latitude and longitude lines. The macrocarrier 60 comprises a horizontal equatorial plate 62 intersected by a series of vertical ribs 64. In addition, secondary horizontal plate 66a, 66b are located approximately midway between the equatorial plate 62 and the top and bottom poles of the macrocarrier 60. The secondary horizontal plates 66a, 66b generally mimic the Tropic of Cancer and Tropic of Capricorn latitude planes found on a globe. As in the first embodiment, there are three vertical ribs 64 such that the entire macrocarrier 60 structure defines twenty-four discrete spaces or interstices. As in the earlier embodiments, the plates and ribs 62-66 of the macrocarrier 60 define a plurality of flat surfaces; namely, horizontal surfaces 70 on the top and bottom faces of the plates, and vertical surfaces 72 on either side of the ribs.

In contrast to the earlier embodiments, the “Honey Dipper” macrocarrier 60 features a center flow channel 74 extending along the central vertical axis 76. The central flow channel 74 is preferably cylindrical and defined by the inner edges of each of the plates and ribs 62-66, which are therefore not continuous across the width of the macrocarrier 60. Provision of a central flow channel enhances cell growth by permitting the fluid broth carrying the cells greater access to the flat surfaces 70, 72 within the structure of the macrocarrier 60. In a preferred embodiment there are at least 3 vertical ribs, and preferably between 3-33 total ribs and plates, including the 3 horizontal plates 62, 66. Of course, more ribs and plates could be used for larger macrocarriers, such as up to 5 mm in diameter.

It should be understood that various aspects of the macrocarriers 20, 40, 60 described herein can be interchanged, and thus other permutations not illustrated are contemplated. For example, the equatorial plate 62 on the macrocarrier 60 could be formed in a jagged configuration such as the equatorial plate 42 for the macrocarrier 40. Likewise, the secondary horizontal plates 66a, 66b can be provided for either of the first two embodiments of macrocarriers 20, 40. Finally, the central flow channel 74 in the third embodiment of macrocarrier 60 can be provided for the first two embodiments 20, 40.

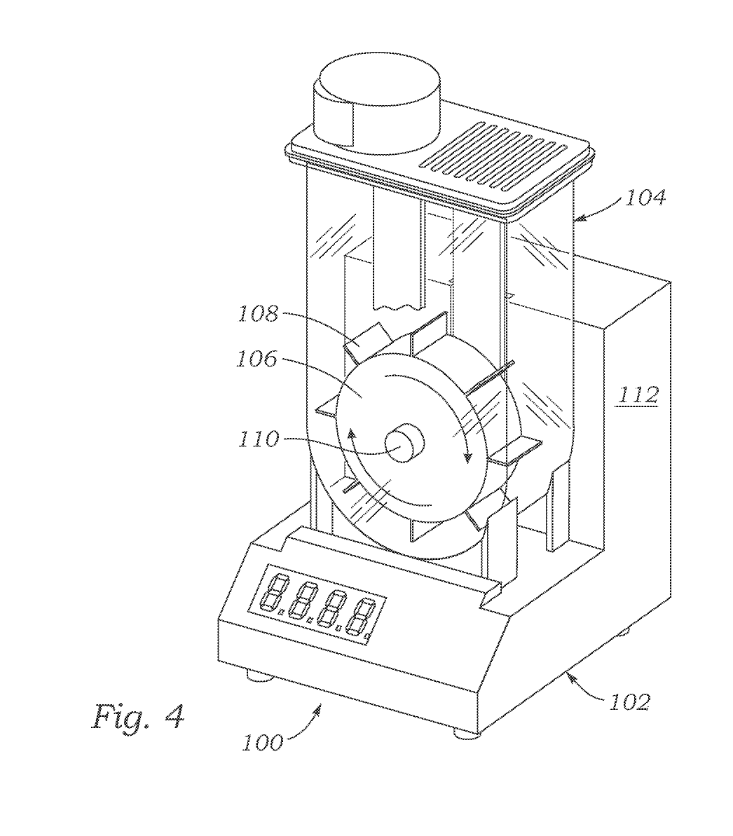

Finally, FIG. 4 illustrates an exemplary embodiment of a small-volume bioreactor 100 in which the macrocarriers described herein can be utilized. The bioreactor 100 comprises a base unit 102 supporting a disposable container 104. The container 104 preferably has a generally rectangular upper section and a semi-cylindrical lower section, as shown. A mixing or agitating wheel 106 is mounted wholly within the container 104 for rotation within the semi-cylindrical lower section. Preferably, the wheel 106 features a series of vanes 108 on its exterior force during the solution within the container 104, and also preferably includes inner vanes (not shown). The wheel 106 rotates about a horizontal axis on hubs 110 secured to the front and/or back walls of the container 104 (i.e., only one wheel hub 110 may be secured to the container 104). In a preferred embodiment, the base unit 102 includes an upstanding cabinet 112 within which is housed a drive system including rotating magnets (not shown). Corresponding magnets or ferromagnetic material mounted around the wheel 106 allow coupling of the drive system to enable rotation of the wheel from outside the container 104, thus eliminating seals and the like which might contaminate the solution within the container. In a preferred embodiment, the volume capacity of the container 104 is between 0.05-1.0 L, although the system can be scaled up for larger capacities.

The illustrated bioreactor 100 is for use inside CO₂ incubators, which are typically run with temperature control and with a fixed percentage of CO₂ in air. Consequently, independent pH and DO controls for the bioreactor 100 are not necessary.

Contemplated options for the macrocarriers include:

The possibility for uneven distribution of mass in order to encourage certain orientation or behavior;

An optimal number/thickness of plates or ribs for different diameters of macrocarrier; and

The inclusion of a center flow channel depending on the macrocarrier diameter.

It is understood that the foregoing examples are considered illustrative only of the principles of the invention. Further, since numerous modifications and changes will readily occur to those skilled in the art, it is not desired to limit the invention to the exact construction and operation shown and, accordingly, all suitable modifications and equivalents may be resorted to, falling within the scope of the invention.

What is claimed is:

1. A macrocarrier for a cell culture growth process, comprising:
a generally ball-shaped bead having an outer size of between about 1-5 mm and defining a vertical polar axis and an equatorial plane, the bead being formed by at least one generally horizontal plate and a series of vertical ribs that are aligned through the polar axis, the plate and ribs defining on opposite faces thereof a plurality of flat surfaces on which cell growth may occur.

2. The macrocarrier of claim 1, wherein there is a single horizontal equatorial plate.

3. The macrocarrier of claim 1, wherein the generally horizontal plate is located approximately in the equatorial plane and comprises a series of differently angled segments between each of the ribs that cross over the equatorial plane.

4. The macrocarrier of claim 1, wherein there are a plurality of generally horizontal plates, a first plate being located approximately at the equatorial plane, and at least one secondary plate being located above the first plate and at least one secondary plate being located below the first plate.

5. The macrocarrier of claim 1, wherein there are between 3-30 vertical ribs.

6. The macrocarrier of claim 1, wherein each of the vertical ribs has a greater thickness at the equatorial plane than at either of opposite poles of the macrocarrier.

7. The macrocarrier of claim 1, further including a central flow channel defined by inner edges of the plate and ribs.

8. The macrocarrier of claim 1, further including a surface treatment on the plurality of flat surfaces to enhance cell growth.

9. The macrocarrier of claim 8, wherein the surface treatment a coating of collagen.

10. The macrocarrier of claim 8, wherein the surface treatment is chosen from the group consisting of:
gas plasma treatment, and
corona discharge treatment.
